(19) United States
(12) Patent Application Publication
(10) Pub. No.: US 2007/0102010 A1
(43) Pub. Date: May 10, 2007

(54) Naso-pharyngeal tissue engineering

(76) Inventors: Stefan M. Lemperle, La Jolla, CA (US); Gottfried H. Lemperle, La Jolla, CA (US); Christopher J. Reinhard, Rancho Santa Fe, CA (US)

Correspondence Address: HELLER EHRMAN LLP, 4350 LA JOLLA VILLAGE DRIVE #700, 7TH FLOOR, SAN DIEGO, CA, 92122, US

(21) Appl. No.: 11/546,578

(22) Filed: Oct. 10, 2006

Related U.S. Application Data
(60) Provisional application No. 60/724,496, filed on Oct. 7, 2005.

(51) Int. Cl. A61M 21/02

(52) U.S. Cl. 128/898; 600/26

(57) ABSTRACT

Methods to stiffen nasopharyngeal structures such as the soft palate, the anterior palatine arch, the posterior palatine arch, or a combination thereof are provided. In one embodiment, the present invention involves the injection, through a conventional needle, of PMMA microspheres that induce a fibrotic response to strengthen and stiffen nasopharyngeal structures. Methods of the invention are useful for the treatment for upper airway conditions such as snoring, obstructive breathing and sleep apnea in a subject.

### REFERENCE TO PRIORITY DOCUMENT

This application claims the benefit of priority of U.S. Provisional Patent Application Ser. No. 60/724,496 entitled “Naso-Pharyngeal Tissue Engineering” by Stefan M. Lemperle, Gottfried H. Lemperle and Christopher J. Reinhard, filed Oct. 7, 2005, herein incorporated by reference.

### FIELD OF THE INVENTION

The field of the invention relates generally to a method of naso-pharyngeal tissue engineering. Embodiments of the invention are for treating upper airway conditions of the naso-pharyngeal area such as snoring and sleep apnea. More particularly, the disclosed invention pertains to a tissue engineering method to stiffen the soft palate, anterior palatine arch, posterior palatine arch, or combination thereof, by injecting biocompatible, non-biodegradable, round and smooth microspheres without substantially impairing other functions of the soft palate such as during speech or swallowing.

### BACKGROUND OF THE INVENTION

The American Academy of Otolaryngology has estimated that 45% of normal adults snore at least occasionally, and 25% are habitual snorers. Snoring occurs when there is an obstruction of the free flow of air through the passages at the back of the mouth and the nose. Snoring is often times caused by vibration of the soft palate tissue when airflow is required to be more forceful as the airway is narrowed.

Obstructive sleep apnea is a disorder in which complete or partial obstruction of the airway during sleep causes loud snoring, oxyhemoglobin desaturation and frequent arousals. Sleep apnea has serious health consequences and can potentially be life threatening. In addition, sleep deprivation resulting from sleep apnea affects both sleepers and their bed partners. The effects of sleep deprivation are daytime sleepiness, a compromised immune system and slower healing, poor mental and emotional health, decreased productivity, negative mood swings and irritability, low energy, unclear thinking and lack of concentration and a slower reaction time.

Dental appliances or oral devices have been developed to treat sleep apnea or snoring. However, these devices are often bulky, cause discomfort and themselves sometimes interfere with sleep patterns. In addition, patient compliance or use of the device is often times poor. Currently there are several surgical treatments for treating snoring and sleep apnea, however these are typically invasive procedures that sometimes require hospitalization. Moreover, few of these methods stiffen the soft palate and, of these, none accomplish stiffening of the soft palate without substantial scarring and inconvenience.

Uvulopalatopharyngoplasty (UPPP) is a surgical procedure for the treatment of severe obstructive sleep apnea. In UPPP, soft tissue on the soft palate and uvula is removed. The tonsils and possibly other excess tissue may also be removed, if present. The procedure increases the width of the airway at the throat opening, improves the opening ability by interrupting muscular action, and “squares off” the palate to enhance its movement and closure. Surgeons usually use either conventional scalpel techniques or newer laser methods (LAUP, or Laser-Assisted Uvulopalatoplasty). LAUP may have a higher rate of success than UPPP, but it also requires the expertise of a surgeon highly skilled in laser procedures. Radiofrequency ablation utilizes a needle electrode to emit energy to shrink excess tissue in the upper airway, including the palate and uvula, the base of the tongue and nasal turbinates. Either way, UPPP generally requires three to five separate treatments, as only a small amount of tissue may be removed at a time. Typically, UPPP and LAUP are approximately 50% to 65% effective in treating sleep apnea. Adverse effects of UPPP include pain, bleeding, infection risk and changes in voice frequency.

U.S. Pat. No. 6,634,362 issued to Conrad et al. describes a method and apparatus for treating snoring involving the precise placement of an implant surgically embedded in the soft palate to alter the dynamic response. In one aspect the implant is a strip that may have a length of about 20-30 mm, a thickness of about 2-4 mm and a width of about 5-10 mm. The strip is implanted by making incisions into the soft palate and inserting the implant into the incision site. In another aspect, the implant has multiple fibers braided or twisted along the length of the implant. In preferred embodiments, the implant is composed of two different fibers: one of non-resorbable material that induces a fibrotic response and the other bio-resorbable or a non-resorbable that provides added stiffness.

Conrad also disclose implanting titanium spheres of approximately 2-4 mm. in diameter so the surgeon may progressively increase the number of implanted modules until the altered dynamic is such that oscillation of the soft palate is abated in normal airflow. U.S. Pat. No. 6,742,524 issued to Knudson discloses the use of a particulate material selected for limited migration within tissue and for encouraging a fibrotic response. A bolus of the particulate material is injected into the tissue area to structurally stiffen the tissue. Knudson further described the use of vitreous carbon, zirconia (ZRO₂), alumina (AL₂O₃) or polymeric particles. It also states that the particles can be carried in a liquid or gel medium. Such particulate matter can cause tissue damage and adverse effects such as foreign body reactions if not biocompatible and round and smooth on the surface, and result in significant scarring.

Restore Medical has recently introduced a snoring treatment called the Pillar™ system. Three braided non-resorbable Polyethylene Terephthalate (PET) inserts, approximately 18 mm in length and 1.5 mm in diameter, are placed parallel front to back in the soft palate (midline, right and left lateral) starting at the hard palate-soft palate juncture during a single treatment session. Clinical trials showed that 22% of treated patients did not respond to such treatment based on measures of snoring intensity and bed partner satisfaction. Scarring at the incision site can cause translocation of the implant. Potential adverse effects include implantation failure, perforation, and discomfort.

Consequently, there is a need for more effective methods to treat snoring and sleep apnea that reduce subject inconvenience and discomfort and limit tissue damage.

### SUMMARY OF THE INVENTION

The present invention is directed towards methods that stiffen nasopharyngeal structures such as the soft palate, the anterior palatine arch, the posterior palatine arch, or a combination thereof. In one embodiment, the present invention involves the injection, through a conventional needle, of PMMA microspheres that can have diameters between 25 and 200 microns (μm), or 25 to 100 microns, or 30 to 50 microns, which are first suspended in a collagen solution and which upon injection induce a fibrotic response within the injected area to strengthen and stiffen the soft tissue in the soft palate, the anterior palatine arch, the posterior palatine arch, or combination thereof. Methods of the invention are useful for the treatment for upper airway conditions such as snoring, obstructive breathing and sleep apnea in a subject.

### DETAILED DISCLOSURE OF THE INVENTION

Further features and advantages of the present invention will become apparent from the detailed description of preferred embodiments which follows.

The palate is the roof of the mouth in vertebrates causing a complete or partial separation of the oral and nasal cavities and consisting of the hard palate and the soft palate. The front part, known as the hard palate, formed by the upper maxillary bones and the palatine bones, separates the oral from the nasal cavity. It is composed of a bone plate covered with a layer of mucous membrane tissue. The back portion, or soft palate, consists of muscular tissue and mucous membranes forming a partial partition between the nasal cavity and the throat. A small cone-like projection, the uvula, hangs from the middle of the soft palate in humans and has little functional value.

Below the soft palate, the pharyngeal wall defines the throat passage. A nasal passage connects the nasal cavity to the pharyngeal walls. The soft tissue which defines the nasal cavity posteriorly is the choana. The nasal cavity, oral cavity and throat passage are collectively referred to as the naso-pharyngeal area. These body surfaces include outer surfaces of the choana, the upper and lower surfaces of the soft palate and outer surfaces of the palatine arches. Outer surfaces means surfaces exposed to air. Both the upper and lower surfaces of the soft palate are outer surfaces. For the purposes of this disclosure, the soft palate, choana and palatine arches are referred to as naso-pharyngeal structures.

Snoring can result from vibration of any one of a number of surfaces or structures of the naso-pharyngeal area. Most commonly, snoring is attributable to vibration of the soft palate. However, vibratory action of the palatine arches can also contribute to snoring sounds. It is not uncommon for vibratory action from more than one region of the naso-pharyngeal area to contribute to snoring sounds. Sleep apnea can result from partial or full collapse of the naso-pharyngeal walls during sleep.

The snoring sound is generated by impulses caused by rapid obstruction and opening of airways. Huang and Williams utilize a spring-mass model to illustrate oscillation of the soft palate in response to airflow (where the soft palate is the ball B of mass depending by a spring S from a fixed anchor A). J. Appl. Physiol. (1999) 86: 1759-1763. Embodiments of the invention are directed to methods for stiffening the soft palate in order to alter its dynamic response to airflow. Such methods are also applicable to the pharyngeal wall and any combination thereof.

A method of the invention entails stiffening of naso-pharyngeal structures, such as the soft palate, by injection of an alloplastic histocompatible implant. In one aspect, the implant can comprise histocompatible microspheres in a biocompatible suspension agent. The implant can be delivered to the desired region of implantation by injection. Said implant can remain immobilized at the injection site.

An implant can be injected into the soft palate by needle or cannula. The needle can be 27 or 26-gauge. The needle can be larger inner diameter (lower gauge number) to effect more rapid injection or smaller inner diameter (higher gauge number) to minimize pain and bleeding and to inject thinner implants. The gauge of the needle is in part determined by the viscosity of the solution, the geometry desired for the injection site, and the comfort and safety of the subject. For example, the needle can be 28, 29, or 30 gauge. Alternatively, the needle can be, for example, 25, 24, 23, 22, 21, 20, 19 or 18 gauge. The needle length can be 1 inch, but can be as short or long as necessary to achieve the appropriate depth of the injection. The needle can also be curved, so as to provide greater flexibility and accessibility.

The implant can be injected by the tunneling technique as described in Lemperle, G. et al. Dermatol. Surg. (2003) 29(6):573-87 and Cohen and Holmes, Plast. Reconstr. Surg. (2004) 114(4):964-76. The implant can be injected in one or more lines, the width of which can be controlled by the skilled practitioner. The lines of implant can be positioned in any direction relative to the other. The lines of implant can be parallel or curve to the shape of the biological structure into which they are being implanted. For example, the soft palate can be injected with three parallel lines or curves of implant to stiffen the structure. In another embodiment, the lines of implant can, for example, intersect, such as forming a criss-cross pattern, in order to achieve greater stiffening of the soft palate or pharyngeal wall. Curved patterns may also be employed, or any combination of curves or lines. Advantageously, the curved patterns can be formed to approximate the countours of the implanted structure. In addition, the flexibility of microsphere-based implant allow for different depths of injection, for example, providing a deep implant in the front of the soft palate (near the hard palate) and a shallower implant in the back, or vice versa, depending on the treatment plan. Other patterns are readily envisioned by the skilled practitioner.

Owing to the fact that the implant of the invention forms a closely packed arrangement of microspheres that remains fixed at the implantation site, it can also be delivered in any three dimensional form. Such a non-predefined three dimensional shape is hereinafter referred to as a “pod”. The pod can be created in a single implantation event or can be built up by multiple implantation events such as by multiple injections. A pod can be created to conform to the anatomical structure to which it is delivered or the pod can alter the anatomical structure. Also, the pod can be altered or moved by gentle manipulation of the disclosed implant after delivery to the implantation site. The flexibility provided by a microsphere-based implant allows for numerous, flexible and individualized treatment scenarios that allows finely controlled adjustment of implantation site topography of the anatomical structure being treated.

Stiffening of the soft palate provides structure to reduce vibration and snoring. Such structure reduces airway collapse, and consequently provides a treatment for sleep apnea. Consequently, methods of the invention include treatments for snoring, obstructive breathing and sleep apnea by stiffening of the soft palate or one or both of the palatine arches, or any combination thereof, by implantation of an implant.

The implant can be a filler material. Filler materials can be those agents that can temporarily fill a void. Alternatively, filler materials can be long-lasting. Filler materials, such as soft tissue fillers, have a variety of applications for tissue augmentation and tissue bulking. Filler materials include those with adhesive properties and those that can be mixed with adhesive agents.

The implant can be a histocompatible solid in the form of a powder. The solid particles forming the powder can be round and smooth in order to minimize cell or tissue damage and to minimize immune stimulation. The round and smooth particles can be essentially free of impurities. It has been observed that when the solid particles are round and smooth and essentially free of impurities, occurrence of granulomas and other side effects caused by a harmful immune response are reduced or eliminated. The particles can be spherical or oblate spheroid and are referred to as microspheres herein. Other geometric configurations that have a smooth surface to minimize cell or tissue damage and to minimize immune stimulation can be used.

In another embodiment, the microparticles can be porous or have a hollow interior.

The microspheres can be greater than or equal to about 20 pm in average diameter to resist phagocytosis and the lymphatic and blood system from washing away any of the implant microspheres. The microspheres can have a diameter between about 20 μm to about 200 μm, a diameter between about 25 μm to about 100 μm, or a diameter between about 30 μm to about 50 μm.

The microspheres can be an inert, histocompatible material. The microspheres can be glass or polymer. The polymer can be cured and polymerized prior to implantation to reduce toxic or carcinogenic potential of the monomers or cure agents. The inert histocompatible polymer can be an acrylic polymer. The acrylic polymer can be a polymer of methacrylate or one of its esters, including, but not limited to, methyl methacrylate, ethyl methacrylate, n-butyl methacrylate, isobutyl methacrylate, lauryl methacrylate, and 2-ethylhexyl methacrylate or any combination or copolymer thereof. Polymethylmethacrylate is also known as PMMA. Injection of PMMA microspheres that are smooth and round has been demonstrated to provide a long lasting effect in the treatment of facial wrinkles (accessible by hypertext transfer protocol (http://) at www.fda.gov/artefill). The alloplastic histocompatible implant disclosed herein can provide a scaffold to promote connective tissue deposition around the microspheres. The microspheres can also be highly refined to limit any inflammation from smaller particles, and to increase the roundness and smoothness properties of the particles.

The microspheres can be suspended in solution. The suspension agent can be an aqueous or non-aqueous solution. The suspension agent can be of sufficient viscosity to promote the suspension of the microspheres. The suspension can be, for example, up to about 0.1, 0.2, 0.5, 1.0, 2.0, 5.0, 10, 15, 20, 30, 40, 50, 60, 70 or 80% by weight microspheres. The amount of microspheres used is determined in part by other components of the solution, such as the carrier concentration, and the method of implantation, such as injection.

The solution can also contain a polymer, which can be histocompatible, as a carrier. The polymer can be a biopolymer such as collagen or cellulose. Collagen allows for the separation of the microspheres to allow tissue ingrowth (tissue engineering). The collagen can be in many types and forms, or in combinations thereof. For example, collagen can be Type I, II or III. Collagen can be native, denatured or cross linked. The various types and forms of collagen are described generally in Methods in Enzymol. (1982) 82:3-217, Pt. A, the contents of which is herein incorporated reference. For example, collagen can be produced from animal derived tissues such as bovine hides, human tissues such as cadaver skin or human cell cultures or through recombinant methods. The solution can contain a collagen fully dissolved or in suspension. The solution can contain up to about 0.1, 0.2, 0.5, 1.0, 2.0, 2.5, 3.0, 3.5, 4.0, 4.5, 5, 6, 7, 8, 9, 10, 20, 30, 40, 50, 60, 70, or 80% (w/w) collagen content. The amount of collagen content in the solution is in part determined by the resultant viscosity, the percentage of other components such as microspheres and the method of implantation, such as injection.

The solution can be sterilized to prevent microbial growth in the solution and to prevent infection in the subject post implantation. The solution can be sterilized by any method that maintains the integrity of the components. Examples of sterilization processes that can be used singly or in combination include 0.2 micron sterile filtration, gamma irradiation or electron beam irradiation.

The solution can also contain an agent that reduces subject discomfort. The agent can be an anesthetic. The anesthetic can be, for example, Articaine, Bupivacaine, Chloroprocaine, Etidocaine, Levobupivacaine, Lidocaine, Mepivacaine, Prilocaine, Procaine, or Tetracaine. The amount of agent included in the implant required to reduce subject discomfort would be readily determined by the skilled practitioner.

The implant can be a suspension of polymethylmethacrylate microspheres, collagen, a buffer, sodium chloride, and water for injection. The implant product can be ArteFill®, which is a suspension of about 20% PMMA microspheres of about 30 μm to about 50 μm average diameter and about 80% by weight a composition of about 3.5% purified bovine collagen, about 2.7% phosphate buffer, about 0.9% sodium chloride, about 0.3% lidocaine hydrochloride, and about 92.6% water for injection. The bovine collagen can be produced from U.S. calf hides. Said implant is disclosed in Lemperle, G. et al. Dermatol Surg. (2003) 29(6):573-87.

Other tissue fillers materials include, but are not limited to, collagen; polylactic acid, polypropylene, polytetrafluoroethylene (PTFE); hollow cylinder pellets such as disclosed in U.S. Patent Publication No. 2004/210230, entitled “Materials and Methods for Soft Tissue Augmentation”; polysaccharide-based gel such as disclosed in U.S. Patent Publication No. 2004/0047892, entitled “Filler Composition for Soft Tissue Augmentation and Reconstructive Surgery”; polyhydroxyalkanoate materials such as disclosed in U.S. Pat. Nos. 6,585,994 and 6,555,123, entitled “Polyhydroxyalkanoate Compositions for Soft Tissue Repair, and Viscosupplementation”; hyaluronic acid including such as disclosed in U.S. Pat. No. 5,827,937; repetitive protein polymers such as disclosed in U.S. Patent Publication No. 2003/0176355, entitled “Synthetic Proteins for In Vivo Drug Delivery and Tissue Augmentation”; a three-part injectable polymer such as disclosed in U.S. Pat. No. 5,785,642; a two-part injectable polymer such as disclosed in U.S. Pat. No. 6,312,725; keratin such as disclosed in U.S. Pat. No. 5,712,252; ceramic microsphere compositions such as disclosed in U.S. Pat. Nos. 5,922,025 and 6,432,437, and 6,537,574, entitled “Soft Tissue Augmentation Material”; biocompatible tissue-reactive prepolymer such as disclosed in U.S. Pat. No. 6,702,731, entitled “Situ Bulking Device”; cross-linked blood plasma proteins such as disclosed in U.S. Pat. No. 7,015,198, entitled “Materials for Soft Tissue Augmentation and Methods of Making and Using Same”; radiation cross-linked hydrogels such as disclosed in U.S. Pat. No. 6,537,569, entitled “Radiation Cross-Linked Hydrogels”; bioelastomers such as disclosed in U.S. Pat. Nos. 6,533,819 and 6,699,294, entitled “Injectable Implants for Tissue Augmentation and Restoration”; cross-linked water-swellable polymer particles such as disclosed in U.S. Pat. Nos. 6,214,331 and 6,544,503, entitled “Process for the Preparation of Aqueous Dispersions of Particles of Water-Soluble Polymers and Particles Obtained”; and compositions including a pseudoplastic polymer carrier such as disclosed in U.S. Pat. No. 5,633,001, entitled “Composition and a Method for Tissue Augmentation”. Filler materials can be cross-linked or not cross-linked.

A filler material can have a short-term effectiveness, wherein effectiveness is characterized by how long the implant remains at the implantation site, of up to about 6 months, a medium-term effectiveness from 6 months to about 3 years, and/or a long-term effectiveness of about 3 years or more, depending upon the nature of the filler material injected. The filler material can be microparticles. The microparticles can be made at least in part of biological materials, such as, for example, but not limited to, one or more of any type of collagen, hyaluronic acid (e.g., animal derived, human derived and/or tissue/cell culture derived), cells, tissues, organisms, genetically altered or not (e.g., purified cytoskeleton of unicellular and/or multicellular algae and/or other organisms), whether cross-linked or not cross-linked, or made of a synthetic and/or polymeric material, such as, for example, polylactic acid, organic compounds, inorganic compounds, ceramic materials, an acrylic polymer, PMMA, polypropylene, polytetrafluoroethylene (PTFE), and combinations thereof. For the purposes of this disclosure, the terms microparticles and microspheres are used interchangeably.

Filler compositions can include a contrast agent. A contrast agent is a biocompatible (non-toxic) material capable of being monitored by, for example, radiography. The contrast agent can be water soluble or water insoluble. Examples of water soluble contrast agents include metrizamide, iopamidol, iothalamate sodium, iodomide sodium, and meglumine. Iodinated liquid contrast agents include Omnipaque®, Visipaque®, and Hypaque-76®. Examples of water insoluble contrast agents are tantalum, tantalum oxide, barium sulfate, gold, tungsten, and platinum. These are commonly available as particles preferably having a diameter of about 10 microns or less.

The contrast agent can be added to the filler composition prior to administration. Both solid and liquid contrast agents can be simply mixed with a solution of the filler material suspensions or with the solid articles. Liquid contrast agent can be mixed at a concentration of about 10 to 80 volume percent or about 20 to 50 volume percent. Solid contrast agents can be added in an amount of about 10 to 40 weight percent or about 20 to 40 weight percent.

By combining a contrast agent with a filler compositions, the skilled artisan can monitor the implantation procedure and verify placement, degree of bulking, and track potential migration of the filler material over time.

Filler compositions can include compositions with materials that aid in growth or suppress growth of the injected or surrounding tissues. For example such embodiments can include compositions comprising autologous body components and fluids as disclosed in co-owned U.S. Patent Publication No. 2006/0093644 A1, entitled “Methods of Administering Microparticles Combined With Autologous Body Components”, herein incorporated by reference. Alternatively, one can prepare a composition comprising cells and a filler material. Cells can be autogeneic, isogeneic, allogeneic or xenogeneic. Cells can be genetically engineered. The compositions can contain different cell types, which may be chosen to act synergistically, for example, in the formation of tissue. The types of cells include stem cells, which can be fetal stem cells or adult stem cells and can be totipotent, multipotent, or pluripotent. Alternatively, the filler material compositions can include non-mammalian eukaryotic cells, prokaryotic cells or viruses.

Filler compositions can include physiologically buffered salt solutions, water, glycerol and the like, and may be supplemented with, for example, serum, growth factors, hormones, sugars, amino acids, vitamins, metalloproteins, lipoproteins, anti-inflammatory molecules, antibiotics and the like. The filler material can be porous or hollow to accommodate said supplements.

Growth factors include, but are not limited to, transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors. Members of the TGF supergene family include the beta transforming growth factors (for example, TGF-beta 1, TGF-beta 2, TGF-beta 3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)); Inhibins (for example, Inhibin A, Inhibin B); growth differentiating factors (for example, GDF-1); and Activins (for example, Activin A, Activin B, Activin AB).

Growth factors can be isolated from native or natural sources, such as from mammalian cells, or can be prepared synthetically, such as by recombinant DNA techniques or by various chemical processes. In addition, analogs, fragments, or derivatives of these factors can be used, provided that they exhibit at least some of the biological activity of the native molecule. For example, analogs can be prepared by expression of genes altered by site-specific mutagenesis or other genetic engineering techniques.

Although an exemplary embodiment of the invention has been shown and described, many other changes, modifications and substitutions, in addition to those set forth in the above paragraphs, may be made by one having ordinary skill in the art without necessarily departing from the spirit and scope of this invention. All references provided in the instant disclosure are herein incorporated by reference.

What is claimed is:

1. A method for stiffening the nasopharyngeal area of a subject comprising implanting a filler material comprising an acrylic polymer into one or more nasopharyngeal structures consisting of the group: the soft palate, the anterior palatine arch, and the posterior palatine arch.

2. The method of claim 1 wherein the filler material is implanted by injection.

3. The method of claim 2 wherein the injection is performed with a needle with a gauge from about 18 to about 30 gauge.

4. The method of claim 3 wherein the needle is 28, 29 or 30 gauge.

5. The method of claim 3 wherein the needle is 26 or 27 gauge.

6. The method of claim 3 wherein the needle is about 1 inch in length.

7. The method of claim 3 wherein the needle is curved.

8. The method of claim 1 wherein the filler material comprises microparticles.

9. The method of claim 8 wherein the microparticles are about 25 microns to about 200 microns in diameter.

10. The method of claim 8 wherein the microparticles are about 25 microns to about 100 microns in diameter.

11. The method of claim 8 wherein the microparticles are about 30 microns to about 50 microns in diameter.

12. The method of claim 8 wherein the microparticles are round and smooth.

13. The method of claim 8 wherein the particles are porous or hollow.

14. The method of claim 1 wherein the acrylic polymer comprises polymethylmethacrylate.

15. The method of claim 1 wherein the filler material remains at the implantation site for up to about 6 months.

16. The method of claim 1 wherein the filler material remains at the implantation site for more than 6 months to less than about 3 years.

17. The method of claim 1 wherein the filler material remains at the implantation site for about 3 years or more.

18. The method of claim 1 wherein the filler material further comprises collagen.

19. The method of claim 18 wherein the collagen comprises human collagen.

20. The method of claim 18 wherein the collagen comprises bovine collagen.

21. The method of claim 18 wherein the collagen is at least partially denatured or at least partially cross-linked or both.

22. The method of claim 1 wherein the filler material further comprises a contrast agent.

23. The method of claim 22 wherein the contrast agent comprises metrizamide, iopamidol, iothalamate sodium, iodomide sodium, or meglumine.

24. The method of claim 22 wherein the contrast agent comprises Omnipaque®, Visipaque®, or Hypaque-76®.

25. The method of claim 22 wherein the contrast agent comprises tantalum, tantalum oxide, barium sulfate, gold, tungsten, or platinum.

26. The method of claim 22 wherein further comprising monitoring the implant by imaging the contrast agent.

27. The method of claim 1 wherein the filler material is histocompatible.

28. The method of claim 1 wherein the filler material comprises polymethylmethacrylate microspheres, collagen, a buffer, sodium chloride, and water for injection.

29. The method of claim 28 wherein the filler material further comprises one or more anesthetics selected from the group consisting of: Articaine, Bupivacaine, Chloroprocaine, Etidocaine, Levobupivacaine, Lidocaine, Mepivacaine, Prilocaine, Procaine, or Tetracaine.

30. The method of claim 29 wherein the filler material comprises about 20% by weight polymethylmethacrylate microspheres and about 80% by weight of a composition comprising about 3.5% purified bovine collagen, about 2.7% phosphate buffer, about 0.9% sodium chloride, about 0.3% lidocaine hydrochloride, and about 92.6% water for injection.

31. The method of claim 30 wherein the microparticles are about 25 microns to about 200 microns in diameter.

32. The method of claim 30 wherein the microparticles are about 25 microns to about 100 microns in diameter.

33. The method of claim 30 wherein the microparticles are about 30 microns to about 50 microns in diameter.

34. The method of claim 30 wherein the microparticles are round and smooth.

35. The method of claim 1 wherein the filler material is implanted in one or more bolus injections.

36. The method of claim 1 wherein the filler material is implanted by a tunneling technique.

37. The method of claim 1 wherein the filler material is implanted in a line.

38. The method of claim 37 wherein the filler material is implanted in a curve that approximates the shape of the biological structure into which it is implanted.

39. The method of claim 1 wherein the filler material is implanted in two or more lines.

40. The method of claim 39 wherein at least two lines are parallel to each other.

41. The method of claim 40 wherein at least two lines intersect each other.

42. The method of claim 1 wherein the filler material is implanted as a pod.

43. The method of claim 33 wherein the pod is reshaped after injection.

44. The method of claim 1 wherein the subject has an upper airway condition.

45. The method of claim 44 wherein the upper airway defect is flaccidity of one or more of the soft palate, the anterior palatine arch, or the posterior palatine arch.

46. The method of claim 45 wherein the upper airway defect results in snoring, obstructive breathing or sleep apnea.

47. A method for stiffening the nasopharyngeal area of a subject comprising implanting a filler material into one or more nasopharyngeal structures consisting of the group: the soft palate, the anterior palatine arch, and the posterior palatine arch. wherein said filler material comprises one or more of the group consisting of: polylactic acid, polypropylene, polytetrafluoroethylene (PTFE), polyhydroxyalkanoate, hyaluronic acid, a protein polymer, polysaccharide-based gel, radiation cross-linked hydrogel, cross-linked water-swellable polymer, a pseudoplastic polymer, a bioelastomer, a three-part injectable polymer or a two-part injectable polymer.

48. The method of claim 47 wherein the filler material comprises hollow cylinder pellets.

49. The method of claim 47 wherein the protein polymer comprises keratin, cross-linked blood plasma protein, or a repetitive protein polymer.

50. A method of treating snoring, sleep apnea or obstructive breathing comprising providing a long-lasting implant comprising polymethylmethacrylate microspheres, collagen, a buffer, sodium chloride, and water for injection and injecting said implant into one or more nasopharyngeal structures consisting of the group: the soft palate, the anterior palatine arch, and the posterior palatine arch.

51. The method of claim 50 wherein the long-lasting implant further comprises one or more anesthetics selected from the group consisting of: Articaine, Bupivacaine, Chloroprocaine, Etidocaine, Levobupivacaine, Lidocaine, Mepivacaine, Prilocaine, Procaine, or Tetracaine.

52. The method of claim 51 wherein the long-lasting implant comprises about 20% by weight polymethylmethacrylate microspheres and about 80% by weight of a composition comprising about 3.5% purified bovine collagen, about 2.7% phosphate buffer, about 0.9% sodium chloride, about 0.3% lidocaine hydrochloride, and about 92.6% water for injection.

53. The method of claim 50 wherein the microparticles are about 25 microns to about 200 microns in diameter.

54. The method of claim 50 wherein the microparticles are about 25 microns to about 100 microns in diameter.

55. The method of claim 50 wherein the microparticles are about 30 microns to about 50 microns in diameter.

56. The method of claim 50 wherein the microparticles are round and smooth.

57. The method of claim 50 wherein the particles are porous or hollow.

58. The method of claim 50 wherein the collagen comprises human collagen.

59. The method of claim 50 wherein the collagen comprises bovine collagen.

60. The method of claim 50 wherein the collagen is at least partially denatured or at least partially cross-linked or both.

61. The method of claim 50 wherein the filler material further comprises a contrast agent.

62. The method of claim 61 wherein the contrast agent comprises metrizamide, iopamidol, iothalamate sodium, iodomide sodium, or meglumine.

63. The method of claim 61 wherein the contrast agent comprises Omnipaque®, Visipaque®, or Hypaque-76®.

64. The method of claim 61 wherein the contrast agent comprises tantalum, tantalum oxide, barium sulfate, gold, tungsten, or platinum.

65. The method of claim 61 wherein further comprising monitoring the implant by imaging the contrast agent.

66. The method of claim 50 wherein the filler material is histocompatible.
